(19) United States
(12) Patent Application Publication
(10) Pub. No.: US 2004/0005608 A1
(43) Pub. Date: Jan. 8, 2004

(54) Sample preparation method and device

(76) Inventors: Michael Saghbini, San Diego, CA (US); Syed F.H. Rehan, Vista, CA (US)

Correspondence Address: Licata & Tyrrell P.C., 66 E. Main Street, Marlton, NJ, 08053, US

(21) Appl. No.: 10/428,887

(22) Filed: May 2, 2003

Related U.S. Application Data
(60) Provisional application No. 60/377,770, filed on May 2, 2002.
(60) Provisional application No. 60/391,462, filed on Jun. 24, 2002.

(51) Int. Cl. C12Q 1/68

(52) U.S. Cl. 435/6

(57) ABSTRACT

A device for biological sample preparation having at least one rod support base, at least one rotatively mounted crusher rod, a sample well support means, and a filter plate is provided. The device of the present invention may be coupled with an analytical measurement device in a system. Also provided is a method for biological sample preparation using the device.

This application claims the benefit of priority from U.S. Provisional Application No. 60/377,770, filed May 2, 2002 and U.S. Provisional Application No. 60/391,462 filed Jun. 24, 2002.

### FIELD OF THE INVENTION

This invention relates to purification processes for nucleic acids and proteins from biological samples without sample transfer and centrifugation steps.

### BACKGROUND OF THE INVENTION

The first step in the purification process of a biological sample is tissue homogenization. Tissue homogenization is normally accomplished enzymatically, mechanically or by a combination of techniques. Enzymatic means include digestion with enzymes. Enzymes weaken tissue to the point of disintegration. However, they require long incubation times ranging from two hours to overnight. Mechanical means include disruption of cell walls by the classical mortar and pestle, using electrical benders with rotating blades or by vigorous shaking in the presence of an abrasive matrix. Shaking devices are complex motor driven units that require tight sample closure during disruption to prevent leaks. Units are available that can process individual tubes, multiple cells or up to 96 well blocks. Following tissue disruption current purification techniques require the removal of cell debris by either centrifugation or filtration. The clear crude lysate is purified using solid phase chemistry in single or multiwell format. Transfer to solid phase columns is done either manually with pipettes or by stacking two filter plates on top of each other. High throughput robotic systems equipped with robotic arms are available for purification from clear crude lysate using vacuum driven or magnetic processes. Generating this starting material is the rate-limiting factor for a complete automated sample preparation process starting from tissues or small organisms.

Crushing samples with a pestle, the simplest and oldest form of tissue homogenization, has been used for decades.

U.S. Pat. No. 6,200,533 discloses a solid phase extraction plate, which includes a unitary tray having a plurality of spaced apart chambers molded therein with each chamber having a top opening and a bottom nozzle with sidewalls.

U.S. Pat. No. 5,906,796 discloses a solid phase extraction plate including a unitary tray having a plurality of spaced-apart discrete upstanding chambers molded therein.

WO 99/06148 discloses a solid phase extraction plate including a unitary tray having a plurality of spaced apart discrete chambers molded therein with each chamber having a top opening and a bottom nozzle.

U.S. Pat. No. 5,795,976 discloses a method for separating heteroduplex and homoduplex DNA molecules in a mixture. The sample mixture is then eluted with a mobile phase containing an ion-pairing reagent and an organic solvent.

U.S. Pat. No. 5,585,236 discloses nonporous polymer beads having an average diameter of about 1-100 microns suitable for chromatographic separation of mixtures of nucleic acids when the polymer beads are alkylated.

U.S. Pat. No. 5,633,129 discloses a method for resolving double stranded DNA species differing by at least one base pair. Each of the species is characterized by an iso-melting domain with a unique melting temperature contiguous with a melting domain of higher thermal stability.

The present invention provides improved methods and devices for preparing samples to be analyzed by high pressure liquid chromatography (HPLC), particularly DHPLC and other chromatographic methods.

### SUMMARY OF THE INVENTION

An object of the present invention is to provide a device for sample preparation comprising a rod support base having a top and bottom side; at least one crusher rod having two ends; a means for mounting one end of the crusher rod rotatably upon the bottom side of the rod support base; a sample well support base having at least one sample well aligned so that the unmounted end of the crusher rod may be inserted into the sample well; and at least one filter plate located inside the sample well and in proximity to the bottom end of the crusher rod. The device of the present invention may be coupled with an analytical measurement device in a system.

A further object of the present invention is to provide a method of preparing biological samples to be analyzed by chromatographic methods comprising loading a filter plate into the sample well of the device and then transferring a biological sample to the filter plate. The filter plate retains and collects DNA from the biological sample. The filter plate is placed on top of the vacuum housing and a crusher rod transported to the filter plate is used to homogenize the biological sample. A vacuum pressure is applied to the filter plate, which draws the biological sample through the filter plate allowing the lysate to be collected in a waste tray. A wash solution is added to the collected DNA from the biological sample to remove impurities so that the biological samples may be analyzed. An elution solution is added to the biological sample and a vacuum is applied to collect DNA from the biological sample into a multiwell collection plate.

### BRIEF DESCRIPTION OF DRAWINGS

FIG. 1 shows a side view of a device for sample preparation.

### DETAILED DESCRIPTION OF THE INVENTION

The present invention provides a device for biological sample preparation and a method for analyzing a plurality of samples. The present invention combines the use of a sturdy multiwell precision crusher with multiwell filter plate technology. A method of purification chemistry, sample preservation, and cell debris trapping process are combined to develop a single plate purification system that does not require any centrifugation or transfer steps. The same filter plate is used to homogenize samples, trap cell debris, to wash away impurities, and to prepare the biological sample.

The device of the present invention comprises a means for homogenizing biological samples to release DNA from the samples; a means for washing away impurities from the homogenized samples; a means for retaining said DNA so that the DNA is collected; a means for releasing said DNA from the retaining means. The device may further comprise a means for analyzing the DNA collected.

A biological sample is placed within the sample well of the multiwell device. A crusher rod is inserted into each chamber and is preferably rotated in a plane that is perpendicular to the long axis of the crusher rod in order to crush the sample. The sample in the sample well is then prepared by crushing the sample between a homogenization device and a filter plate wherein the filter plate may comprise glass fiber. A preferred homogenization device comprises a rod support base having a top and bottom side; at least one crusher rod having two ends; and a means for mounting one end of the crusher rod rotatably upon the bottom side of the rod support base. The crusher rod preferably has one generally flat end in proximity to the filter. The homogenizing action can be enhanced by making the crusher rod surfaces abrasive and by carefully controlling the diameter of the crusher rod with respect to the sample well diameter to ensure optimal sample trapping under the rods and crushing by rotational movement. Also, the proper choice of material in the filter plate itself can enhance the crushing action and contribute to a clog free operation. The crushing is enhanced by grinding between two solid abrasive surfaces. Depending on the chemistry employed, other biological materials besides DNA, such as proteins, could be recovered.

During the crushing step the sample is sandwiched between the crusher rod and the filter. Even if the sample falls outside of the flat surface of the crusher rod, it is still crushed by the grinding movement by pressing the sample against the wall of the well. Crusher rods, therefore, preferably have the same contour as the wells. The grinding motion is preferred to fully homogenize the samples. It can be in a circular format going clockwise or counterclockwise. It can also include XY movements that are perpendicular to each other, where the long axis of the crusher rod defines the Z axis. This is particularly important for robotic arms processing as current versions can move along an XYZ platform.

Additionally, a depth filter may be used. The depth filter is situated in between the filter plate and the flat end of each crusher rod. The sample to be prepared is preferably positioned between the crusher rod and the depth filter or filter plate.

The means for washing away impurities of DNA can include washing an alcohol solution, preferably isopropanol. In a preferred embodiment the alcohol solution is present at a concentration of not greater than 10%. In a more preferred embodiment the alcohol solution is present at a concentration not greater than 5%. In a most preferred embodiment the alcohol solution is present at a concentration not greater than 2.5%.

The means for retaining DNA can include a binding mechanism for DNA such as silica used in the filter plate in the presence of chaotropic salts. The filter material supports the grinding action and traps tissue and cell debris. In a preferred embodiment the retaining means is a silica filter. This process can be enhanced by placing a depth filter on top of the filter or binding material to provide a solid surface for crushing. The depth filter can be made of a variety of materials and different textures for a more efficient grinding and filtration process. Debris is trapped throughout the depth filter material. Depth filters can consist of layers of membranes with decreasing pore sizes; one such example is EMPORE (http://www.3m.com/empore). Depth filters may also consist of standard porous grits less than 0.1 mm in thickness such as those available from Porex (http://www.porex.com) in fine, medium or coarse formats.

The method also preferably further includes a step for releasing the DNA from the retaining means by adding an elution solution. In a preferred embodiment, the method further includes applying a vacuum for removing liquid from the filter plate. In a further preferred embodiment, the method includes positioning a multiwell collection plate for collecting liquid from said multiwell device. In a yet further preferred embodiment, a multiwell collection plate is positioned for collecting DNA from the biological sample after an elution solution is added to the biological sample. The DNA from the biological sample is then analyzed using an analytical method such as HPLC, ion exchange liquid chromatography or capillary electrophoresis. The sample preparation method and device of the instant invention can be adapted for use with automated liquid handling platforms such as robotic systems. Examples of such robotic systems include those available from Beckman Coulter (Biomek 2000 Workstation, Part No. 6090001, Beckman Coulter, Fullerton, Calif.) or from Hamilton (MicroLab System, Hamilton, Reno, Nev.).

All solid phase DNA binding matrices are hydrophilic in nature causing aqueous based solutions needed for DNA binding and tissue stabilization to drip within minutes. A mechanism to contain the stabilizing solution-submerged samples to preserve biological sample integrity while waiting to be processed may be used. Dripping can be prevented by sealing the dripping tips or column tops with a piercable seal, such as aluminum, paraffin or any other suitable material. A piercing element may be used which comprises single or multiple well fixed piercing rods if the plates are sealed on the bottom or a mobile element if the plates are sealed on the top. Alternatively a hydrophobic layer can be added on top of the DNA binding material such that an active force, such as a vacuum, is needed to pull the liquid through the layer.

The purification chemistry method involves the reversible binding of DNA to silica in the presence of chaotropic salts. The DNA is then washed in an alcohol based solution and eluted in water. The alcohol content of the wash solution is important, it is the alcohol presence that is essential to retain the DNA on the filter while washing away contaminants. Minimizing the amount of alcohol in final DNA eluate is necessary as it inhibits many enzymatic activities including PCR. Traditional wash solutions contain more than fifty percent ethanol. Vigorous tapping on a stack of absorbent paper in addition to vacuum filtration is needed to prevent alcohol from traditional wash solutions from contaminating the final product. The tapping and filtration greatly reduces the throughput. Alcohol amounts in the wash solution of the present invention have been successfully reduced to about 2.5% alcohol allowing removal of this inhibitory substance by vacuum filtration alone. It is further possible to wash with water if the incubation time is limited.

The present invention, using only filter plate purification process has been successfully used to isolate genomic DNA from multiple biological samples. Biological samples include but are not limited to: animal parts and tissues, such as Drosophila and zebra fish body parts; fungi; viruses; bacteria; and plants, such as fresh corn kernels, peas, and green beans. The biological samples may be purified for PCR analysis. The present invention is particularly useful in processing most soft to moderately hard three dimensional type animal and plant tissues. The amount of tissue used is carefully controlled to eliminate clogging.

The device of the present invention may be coupled with an analytical measurement device such as HPLC, capillary electrophoresis device or ion exchange chromatography device to provide a system for biological sample preparation.

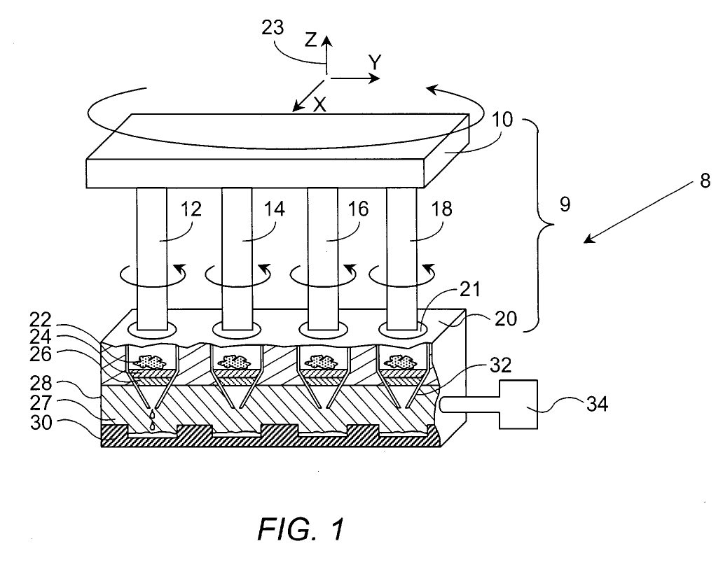

As shown in FIG. 1, the device for biological sample preparation 8 comprises a homogenizing means 9 for homogenizing the samples, and a sample well support base 20. The homogenizing means 9 comprises a rod support base 10 having a top and bottom side, at least one crusher rod 12, 14, 16, 18, and a means for mounting the one end of the crusher rod rotatably upon the bottom side of the rod support base. The sample well support 20 comprises sample wells 21 adapted for receiving the crusher rods 12, 14, 16, 18. The sample well support base 20 having at least one sample well is aligned so that the unmounted end of a crusher rod 12, 14, 16, 18 may be inserted into the sample well and contact a biological sample resting upon at least one filter plate 26 located inside the sample well. At least one filter plate 26 is positioned in each of the sample wells 21 at an appropriate position for filtration such as in the lower portion of the sample wells 21 and in proximity to the bottom end of the crusher rod, so that when a biological sample 22 is placed in the sample well the sample is able to be crushed between the filter 26 and the tip of the crusher rod 12, 14, 16, 18 furthest away from the crusher rod support 10, when the crusher rod 12, 14, 16, 18 are rotated in a plane perpendicular to Z, the long axis of the crusher rod 23 and within the sample wells. A plurality of parallel rods may be arranged to fit insertably within the wells of a multiwell device. The multiwell device may include an array of chambers or wells. In addition to binding DNA, the filter plate 26 must be able to withstand the crushing action and to remove cell debris and hold onto the debris without clogging the filtration process. In a preferred embodiment, DNA binding filter plates are ideally suited for this purpose. Any filter plates with thick silica or glass fiber membranes or packed with silica-like powders are suitable. One example of a preferred filter plate is a solid phase extraction disk (from ANSYS Technologies part no. 596-08. Filter plates that use thin glass fiber membranes cannot be used individually, as the membranes are easily torn during the gentle crushing action. However, the inclusion of a depth filter may be used to allow thin glass fiber membranes to be utilized. Padding filter plates increases the difficulty of eliminating alcohol from traditional wash solutions, but not from the modified wash solutions, which can remove or eliminate alcohol by vacuum filtration alone. A vacuum housing is positioned so that the biological sample may be drawn through the filter plate and collected in a multiwell collection plate. A vacuum source is used for applying a vacuum pressure to the filter plate to draw the biological sample through the filter plate. A vacuum housing 27 and a vacuum source 34 may be positioned to draw liquid 28 through each filter plate into either the waste tray or the multiwell collection plate. Each well 21 is associated with an outlet 32 or dripping tube adapted for allowing the liquid to flow out of the sample well and into a multiwell collection plate 30 positioned to receive the liquid from each outlet 32. The crusher rods 12, 14, 16, 18 and the sample wells 21 are preferably arranged such that the outlets 32 can drain into the wells of a multiwell collection plate 30 positioned beneath the outlets 32. The filter plate can additionally include a depth filter 24 positioned above the filter plate 26. An example of a depth filter 24 is a 0.45 μm depth filter (ANSYS Technologies part no. 596-00-45). However any other suitable depth filter may be used.

In another embodiment a system for sample preparation and analysis of a plurality of tissue or other samples comprises the sample preparation device 8 and an analytical device such a an HPLC, a capillary electrophoresis device or an ion exchange chromatography device. Examples of such analytical devices include the WAVE DNA analysis system from Transgenomic and the ProStar Helix system from Varian. A one filter plate purification system for processing tissues without sample transfer and centrifugation steps is ideal for high throughput robotics. The crusher rod can be used with robotic arms to homogenize samples via XYZ rotational movements. Plate stacks, commonly referred to as hotels, exist to hold plates for processing and storage. Robot friendly vacuum manifolds are available for filter plate manipulations.

The system may be cleaned by actively washing the device in a cleaning solution. The system or any of its components may be rinsed with tap water and then placed in a sonicating water bath for a length of time, preferably three to five minutes. The pieces may then be dried on a rack, paper towel or other suitable means for drying. If necessary, the components of the system, and particularly the crushing rods can be soaked in a bleach solution for a period of time, such as ten minutes before washing. Alternatively a disposable plastic lining similar to those used as ear thermometer covers may be employed. The process of putting the covers onto the platform and removing the covers from the platform may be accomplished via mechanical or probiotic means.

The present invention further provides a method of preparing biological samples to be analyzed by chromatographic methods comprises loading a filter plate into the sample well of the biological sample preparation device described above. The biological sample is transferred to the filter plate. The filter plate retains and collects DNA from the biological sample. The filter plate is moved on top of a vacuum housing. The crusher rod is transported to filter plate where it is inserted into the sample well. The crusher rod is moved rotatively or in a upward and downward manner to homogenize the biological sample. A vacuum pressure is then applied to the filter plate which draws the liquid from the biological sample through the filter plate. The resulting lysate is collected in a waste tray. A wash solution is added to the filter plate to remove impurities from the collected DNA of the biological sample so that the biological samples may be analyzed. The waste tray is then able to be removed and replaced with a multiwell collection plate. An elution solution, preferably ddH2O, is added to the sample. A vacuum is then applied to collect the DNA from the biological sample into the multiwell collection plate. The wash solution may be repeated two or more times and then vacuumed to remove traces of wash. The samples are analyzed using an analytical method such as HPLC, ion exchange liquid chromatography or capillary electrophoresis. The sample preparation system and method of the instant invention can be adapted for use with automated liquid handling platforms such as robotic systems.

As would be understood by one of skill in the art upon reading this disclosure, the dimensions of the components and the recited numbers associated with the various components provided herein are merely exemplary and may be varied routinely by those of skill in the art depending upon the desired application.

The present invention is further illustrated by the following non-limiting examples.

### EXAMPLE 1

### DNA Isolation

Flies and fish tissue samples are directly crushed in a 96 well DNA filter plate (0.2-0.3 ml 96 well plate with skirt (i.e Nunc catalog No. 276-012) in the presence of a DNA Binding and stabilizing solution comprising 3.4M Guanidine Thiocyanate, 15% PEG 8000, 100 mM Tris, 10% IPA and which has a pH of 7.4. The device of the present invention as shown in FIG. 1 was used. A wash solution of 2.5% isopropanol in water was used to wash the samples and remove PCR contaminants. Following a simple washing step, the genomic DNA was eluted in sterile water (ddH₂O)and was ready for PCR. All steps are done in a vacuum manifold. No centrifugation is required. 96 preps were purified in about 45 minutes with many plates processed in a single working day.

### EXAMPLE 2

### DNA Binding

The DNA binding plate was placed on a parafilm sheet on a bench-top and gently rotated back and forth few times to seal the dripping tips. 300 μl of DNA Binding and stabilizing solution was added per well to be used. The samples were fully submerged to preserve genomic DNA integrity. The samples were then homogenized by placing the samples onto the binding plate and rotating clockwise about 10 times. The samples were incubated for about 5 minutes at room temperature. The crusher rods were sized to cover more than ¾ of the well surface area. The DNA filter plate was assembled with the crusher rod still in place into the Vacuum manifold containing the waste tray in the bottom position and vacuumed until all the wells are dry (about three to five minutes). The crusher rods were removed, and 300 μl wash solution was added. Then a vacuum was applied until all the wells were dry. 300 μl wash solution was added and a vacuum was applied for two to three minutes. The wash step was repeated one more time. The binding plate was dried for 10 minutes under vacuum. The waste tray was removed and replaced with a collection plate. 150 μl of elution solution (ddH2O) was added and incubated for about 5 minutes at room temperature. A vacuum was applied for 5 minutes to collect the DNA into the collection plate. The DNA was then ready to use. The plate was sealed with adhesive seal and stored at −20 C. 2.5 μl of genomic DNA was used for a 50 μl PCR reaction.

What is claimed is:

1. A device for sample preparation comprising:
(a) a rod support base having a top and bottom side;
(b) at least one crusher rod having two ends;
(c) a means for mounting one end of the crusher rod rotatably upon the bottom side of the rod support base;
(d) a sample well support base having at least one sample well aligned so that the unmounted end of the crusher rod may be inserted into the sample well; and
(e) at least one filter plate located inside the sample well and in proximity to the bottom end of the crusher rod so that a biological sample may be transferred to the filter plate, wherein the filter plate retains, collects and releases DNA from the biological sample.

2. The device of claim 1 wherein the sample well base further comprises:
(a) a vacuum housing positioned so that a biological sample may be drawn through the filter plate and collected in a multi-well collection plate; and
(b) a vacuum source for applying a vacuum pressure to the filter plate to draw the biological sample through the filter plate.

3. The device of claim 1 wherein the crusher rod rotates in a plane perpendicular to the long axis of the crusher rod.

4. The device of claim 1 further comprising a depth filter positioned in between the filter plate and the unmounted end of the crusher rod.

5. A system for sample preparation comprising the device of claim 1 and an analytical measurement device.

6. A method of preparing biological samples to be analyzed by chromatographic methods comprising:
(a) loading a filter plate into the sample well of the device of claim 2;
(b) transferring a biological sample to the filter plate wherein the filter plate retains and allows collection of DNA from the biological sample;
(c) moving the filter plate on top of the vacuum housing;
(d) transporting a crusher rod to the filter plate;
(e) moving the crusher rod to homogenize the biological sample;
(f) applying a vacuum pressure to the filter plate which draws the biological sample through the filter plate;
(g) collecting the lysate in a waste tray;
(h) adding a wash solution to collected DNA from the biological sample to remove impurities so that the biological samples may be analyzed;
(I) removing the waste tray and replacing the waste tray with a multiwell collection plate;
(j) adding an elution solution; and
(k) applying a vacuum to collect DNA from the biological sample into the multiwell collection plate.

7. The method of claim 6 wherein the wash solution is repeated two times and then vacuumed to remove traces of wash.
